(19) United States
(12) Patent Application Publication
(10) Pub. No.: US 2006/0052024 A1
(43) Pub. Date: Mar. 9, 2006

(54) Liquid absorbent sheet and method for manufacturing the same

(75) Inventors: Akira Shibata, Kagawa (JP); Satoshi Nozaki, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/186,235

(22) Filed: Jul. 21, 2005

(30) Foreign Application Priority Data
Sep. 7, 2004 (JP) 2004-259789

(51) Int. Cl. D04H 13/00; A61F 13/15; B32B 5/26; D04H 1/54

(52) U.S. Cl. 442/389; 442/327; 442/409; 442/381; 604/358

(57) ABSTRACT

Disclosed is a liquid absorbent sheet for absorbing and retaining liquid. The liquid absorbent sheet is a single nonwoven fabric in which a liquid permeable layer and a liquid retaining layer are inseparably but distinguishably formed. The liquid permeable layer is exposed externally on one side of the nonwoven fabric. The liquid retaining layer lies beneath the liquid permeable layer for absorbing and retaining liquid. The liquid permeable layer contains at least 70% synthetic resin fibers by weight. The liquid retaining layer contains at least 70% cellulosic fibers by weight. Constituent fibers of the nonwoven fabric are bonded together by an adhesive force of a binder and/or a bonding force caused by fusion of the synthetic resin fibers.

### BACKGROUND OF THE INVENTION

1. Field of the Invention

The present invention relates to a liquid absorbent sheet intended for use in absorbing a drip exuding from foods or intended for other liquid absorbing applications.

2. Description of the Related Art

Liquid absorbent sheets have been used for various applications. Typically, such liquid absorbent sheets are constructed to include cellulosic fibers, such as pulp and rayon, for retaining water between fibers due to hydrophilicity of fibers. In liquid absorbent sheets of this type, an additional layer is preferably provided on a side to which liquid is to be applied so as to prevent liquid, which has been absorbed in the liquid absorbent sheet, from coming into direct contact with a liquid discharging object.

Japanese Unexamined Patent Application Publication Nos. H08-119341, H10-166485 and 2002-300848 disclose liquid absorbent sheets that are intended to be laid on a food tray or the like for absorbing a drip, such as blood, exuding from fresh foods, such as meat and fresh fish. More particularly, they disclose an apertured resin film that is laid on and bonded to a liquid retaining layer made of a hydrophilic material. With a fresh food laid on the resin film, the drip exuding from the fresh food can pass through the apertures of the resin film and be then absorbed and retained by the liquid retaining layer. Since the resin film is interposed between the liquid retaining layer and the fresh food, the drip retained in the liquid retaining layer is effectively prevented from returning and adhering to the fresh food, thereby keeping the fresh food from discoloring or deteriorating.

On the other hand, Japanese Unexamined Patent Application Publication No. 2004-105349 discloses a liquid absorbent sheet suitable for use in a thin absorbent article such as panty liner. This liquid absorbent sheet is constructed such that a first sheet, in which synthetic resin fibers are fusion-bonded together by heat, is laid on a second sheet, in which cellulosic fibers and synthetic resin fibers are entangled together, and the first and the second sheets are bonded together by a bonding force caused by fusion of the synthetic resin fibers. When the liquid absorbent sheet is used with the first sheet directed to a liquid receiving side, liquid discharged from a wearer's body moves to the second sheet by capillary action between fibers of the first sheet and is then absorbed and retained by the second sheet. With the first sheet interposed between the second sheet and the wearer's body, the liquid retained in the second sheet is effectively prevented from returning and adhering to the wearer's body.

In the liquid absorbent sheets disclosed in Patent Application Publication Nos. H08-119341, H10-166485 and 2002-300848, however, since there is a limit to increasing the percent open area of the resin film that is exposed externally on the liquid receiving side, a part of liquid applied to the surface of the apertured resin film may be left thereon without transferring to the liquid retaining layer. In addition, since a step of laminating the resin film to the liquid retaining layer is required in the process of manufacturing the liquid absorbent sheet, the manufacturing process becomes complicated to increase the unit cost.

In the liquid absorbent sheet disclosed in Patent Application Publication No. 2004-105349, on the other hand, voids between the synthetic resin fibers constituting the first sheet can serve as liquid passages. Therefore, the first sheet is more effective in preventing liquid from remaining on the sheet surface than the apertured resin film. However, since the first and the second sheets need be joined together along a boundary therebetween, the manufacturing process requires a joining step and becomes complicated. In addition, since the boundary of the two sheets, in which a molten resin for joining the two sheets is concentrated, may resist and delay transfer of liquid from the first sheet to the second sheet.

### SUMMARY OF THE INVENTION

The present invention has been developed to solve the problems in the prior art set forth above and has an object to provide a liquid absorbent sheet which permits liquid, which is applied to a liquid receiving surface thereof, to rapidly transfer to a liquid retaining layer and is also effective in preventing the liquid from returning from the liquid retaining layer to the liquid receiving surface.

Another object of the present invention is to provide a method for manufacturing the same.

According to a first aspect of the present invention, there is provided a liquid absorbent sheet for absorbing and retaining liquid, the liquid absorbent sheet being a single nonwoven fabric in which a liquid permeable layer and a liquid retaining layer are inseparably but distinguishably formed, the liquid permeable layer being exposed externally on one side of the nonwoven fabric, the liquid retaining layer lying beneath the liquid permeable layer for absorbing and retaining liquid,
the liquid permeable layer containing at least 70% synthetic resin fibers by weight, the liquid retaining layer containing at least 70% cellulosic fibers by weight,
constituent fibers of the nonwoven fabric being bonded together by an adhesive force of a binder and/or a bonding force caused by fusion of the synthetic resin fibers.

In the liquid absorbent sheet according to the present invention, since the liquid permeable layer is mainly formed of synthetic resin fibers, liquid retained in the liquid retaining layer is effectively prevented from returning to the liquid receiving surface. In addition, since the liquid permeable layer and the liquid retaining layer are inseparably but distinguishably formed in the single nonwoven fabric without the need of joining separate sheets, the liquid absorbent sheet can be easily manufactured and liquid can smoothly transfer from the liquid permeable layer to the liquid retaining layer across a boundary therebetween.

According to one embodiment of the present invention, the liquid retaining layer may lie within the nonwoven fabric with two liquid permeable layers exposed externally on opposite sides of the nonwoven fabric. When this liquid absorbent sheet is laid on a food tray, because one liquid permeable layer is interposed between a fresh food and the liquid retaining layer, a drip retained in the liquid retaining layer is effectively prevented from returning and adhering to the fresh food on the liquid absorbent sheet. Furthermore, because the other liquid permeable layer is interposed between the tray surface and the liquid retaining layer, the drip retained in the liquid retaining layer is effectively prevented from returning and adhering to the tray surface.

Alternatively, the liquid retaining layer may be exposed externally on the other side of the nonwoven fabric.

Preferably, the liquid retaining layer has a higher fiber density than the liquid permeable layer. With such a difference in fiber density, the liquid passing through the liquid permeable layer can rapidly transfer to the liquid retaining layer, while the liquid retained in the liquid retaining layer is effectively prevented from returning to the surface of the liquid permeable layer.

According to a second aspect of the present invention, there is provided a method for manufacturing a liquid absorbent sheet comprising the steps of:
(a) depositing a multi-ply fibrous web with a liquid permeable fibrous web containing at least 70% synthetic resin fibers by weight and a liquid retaining fibrous web containing at least 70% cellulosic fibers by weight;
(b) pressing the multi-ply fibrous web; and
(c) before or after the step (b), bonding constituent fibers of the multi-ply fibrous web by an adhesive force of a binder and/or a bonding force caused by fusion of the synthetic resin fibers.

According to the above liquid absorbent sheet manufacturing method, the liquid permeable layer and the liquid retaining layer can be inseparably but distinguishably formed in the single nonwoven fabric by a continuous nonwoven fabric manufacturing process without the need of joining separate sheets.

The method may further comprise a step (d) of drying the multi-ply fibrous web after the steps (a), (b) and (c). When a binder is utilized, the drying step enables rapid solidification of the binder.

In the step (a), constituent fibers of the liquid permeable fibrous web and constituent fibers of the liquid retaining fibrous web are preferably deposited by an air-laid process. When deposited by an air-laid process, excessive fiber mixture can be prevented at the boundary between the liquid permeable fibrous web and the liquid retaining fibrous web. In the finished nonwoven fabric after the steps (b) and (c), accordingly, the liquid permeable layer and the liquid retaining layer may be inseparably but distinguishably formed so that they can exhibit different functions in a clearly distinguishable manner.

Also in the step (a), the multi-ply fibrous web may be formed by depositing the liquid retaining fibrous web on the liquid permeable fibrous web and further depositing another liquid permeable fibrous web on the liquid retaining fibrous web to obtain a liquid absorbent sheet with a liquid retaining layer sandwiched between two liquid permeable layers.

### BRIEF DESCRIPTION OF THE DRAWINGS

The present invention will be understood more fully from the detailed description given hereinafter and from the accompanying drawings of the preferred embodiments of the present invention, which, however, should not be taken to limit the invention, but are for explanation and understanding only.

In the drawings:

FIG. 1 is a perspective view of a liquid absorbent sheet according to one embodiment of the invention;

FIG. 2 is an enlarged sectional view of a liquid absorbent sheet according to a first embodiment of the invention;

FIG. 3 is an enlarged sectional view of a liquid absorbent sheet according to a second embodiment of the invention; and

FIG. 4 is an explanatory drawing showing a method for manufacturing a liquid absorbent sheet according to one embodiment of the invention.

### DESCRIPTION OF THE PREFERRED EMBODIMENT

The present invention will be discussed hereinafter in detail in terms of the preferred embodiments according to the present invention with reference to the accompanying drawings. In the following description, numerous specific details are set forth in order to provide a thorough understanding of the present invention. It will be obvious, however, to those skilled in the art that the present invention may be practiced without these specific details. In other instance, well-known structures are not shown in detail in order to avoid unnecessary obscuring of the present invention.

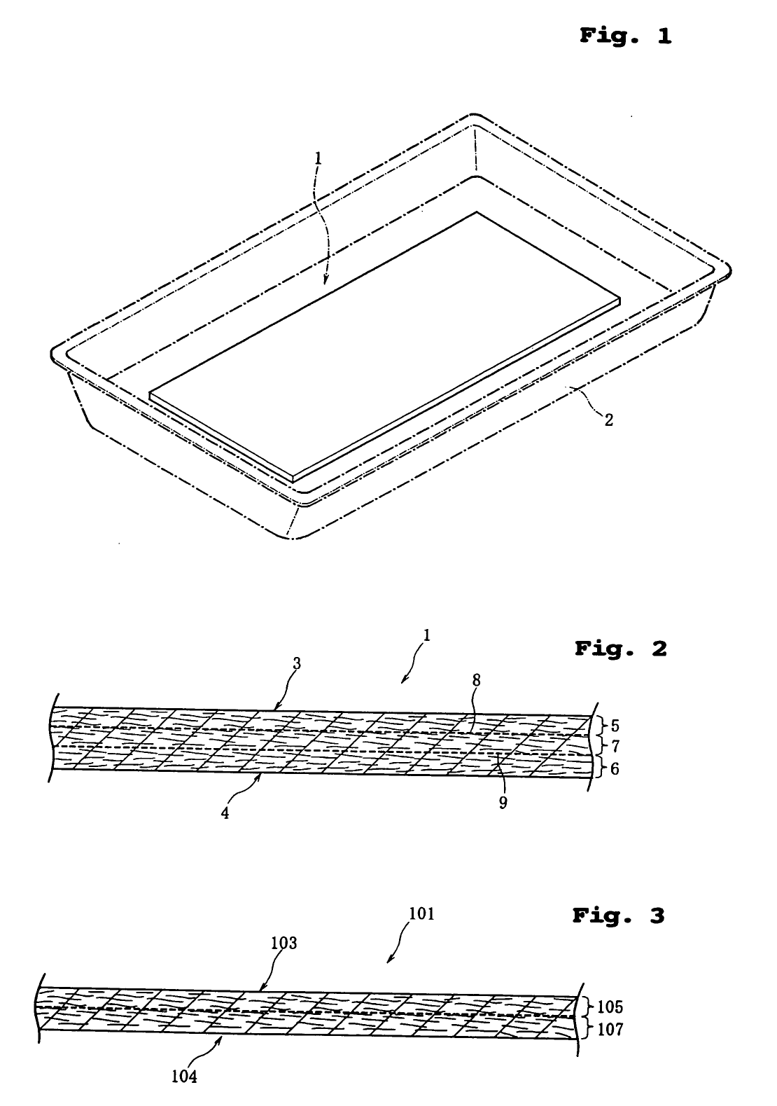

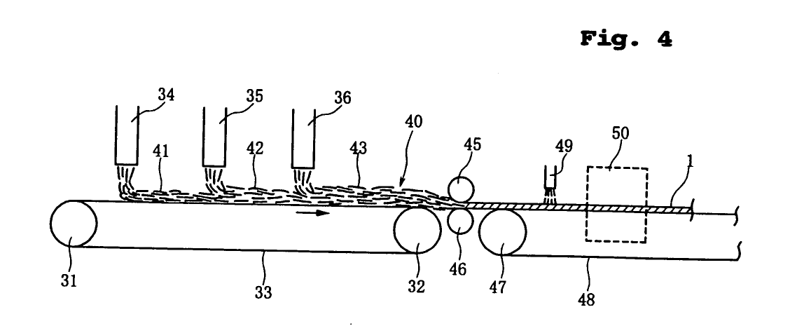

FIG. 1 is a perspective view of a liquid absorbent sheet according to one embodiment of the invention; FIG. 2 is an enlarged sectional view of a liquid absorbent sheet according to a first embodiment of the invention; FIG. 3 is an enlarged sectional view of a liquid absorbent sheet according to a second embodiment of the invention; and FIG. 4 is an explanatory drawing showing a method for manufacturing a liquid absorbent sheet according to one embodiment of the invention.

As shown in FIG. 1, a liquid absorbent sheet 1 may be used on a bottom panel of a food tray 2. The food tray 2 may be formed of a styrofoam or polyethylene (PET) sheet. With a food such as meat, processed meat product, fresh fish, processed fish product, etc. laid on the liquid absorbent sheet 1, the food tray 2 housing the food may be wrapped in a wrapping film and put on the market.

Alternatively, a fresh food such as meat, fresh fish, vegetable, etc. may be directly wrapped in the liquid absorbent sheet 1.

As shown in FIG. 2, the liquid absorbent sheet 1 has first and second surfaces 3, 4 and its sheet thickness is about 0.3 to 3 mm.

The liquid absorbent sheet 1 is a single nonwoven fabric which may be manufactured by a continuous nonwoven fabric manufacturing process as will be described hereinbelow. As shown in FIG. 2, the liquid absorbent sheet 1 may be divided into three layers in a thickness direction. The layer exposed externally on the side of the first surface 3 is a first liquid permeable layer 5, the layer exposed externally on the side of the second surface 4 is a second liquid permeable layer 6, and the layer sandwiched between the liquid permeable layers 5, 6 is a liquid retaining layer 7. The first and the second liquid permeable layers 5, 6 may be of the same fiber makeup, but the liquid retaining layer 7 may have a different fiber makeup from the liquid permeable layers 5, 6.

Although the liquid absorbent sheet 1 is a single nonwoven fabric, a boundary 8 between the first liquid permeable layer 5 and the liquid retaining layer 7 and a boundary 9 between the second liquid permeable layer 6 and the liquid retaining layer 7 are relatively easily recognizable.

The first and the second liquid permeable layers 5, 6 may be composed of 70 to 100% synthetic resin fibers by weight and 0 to 30% cellulosic fibers by weight with a basis weight of at least 12 g/m². The upper limit of the basis weight is not particularly defined, but may be about 60 g/m², for instance. The liquid retaining layer 7 may be composed of 70 to 100% cellulosic fibers by weight and 0 to 30% synthetic resin fibers by weight with a basis weight of at least 10 g/m². The upper limit of the basis weight is not particularly defined, but may be about 50 g/m², for instance. Preferably, the first and the second liquid permeable layers 5, 6 are composed only of synthetic resin fibers and/or the liquid retaining layer 7 is composed only of cellulosic fibers.

Examples of the synthetic resin fibers include PE (polyethylene) fibers, PP (polypropylene) fibers, PET (polyethylene terephthalate) fibers, PE/PE bicomponent fibers, PE/PET bicomponent fibers, and low-melting PP/high-melting PP bicomponent fibers. For the liquid retaining layer 7, the synthetic resin fibers are preferably treated to be hydrophilic with a hydrophilic agent such as a surfactant coated thereon or kneaded therein. For the first and the second liquid permeable layers 5, 6, the synthetic resin fibers are preferably treated to be hydrophilic, but may be used without being treated to be hydrophilic. Moreover, the synthetic resin fibers for the first and the second liquid permeable layers 5, 6 are preferably whitened with a whitening agent such as an inorganic oxide kneaded therein. With the whitened synthetic resin fibers, the color of liquid, such as blood from a food, absorbed in the liquid retaining layer 7 becomes less noticeable from the outside.

Examples of the cellulosic fibers include natural fibers, such as pulp and cotton, and regenerated cellulose fibers, such as rayon.

The liquid absorbent sheet 1 (nonwoven fabric) may be compressed between rolls to smooth the first and the second surfaces 3, 4. Here, the liquid retaining layer 7 has a higher fiber density than the first and the second liquid permeable layers 5, 6.

Constituent fibers of the first and the second liquid permeable layers 5, 6 and constituent fibers of the liquid retaining layer 7 may be bonded together through a binder. The binder is preferably an acrylic emulsion binder or the like. Such an emulsion binder may be applied to at least one of the first and the second surfaces 3, 4 or may be distributed almost uniformly in the individual layers.

Alternatively, the binder may be a fibrous binder of which at least the fiber surface is made of a low-melting point resin. Where constituent fibers of the first and the second liquid permeable layers 5, 6 are not heat-fusible, for example, they may be bonded together through such a fibrous binder. The cellulosic fibers of the liquid retaining layer 7 may also be bonded by adding the fibrous binder into the liquid retaining layer 7. Here, the fibrous binder is included in 70 to 100% synthetic resin fibers by weight of the first and the second liquid permeable layers 5, 6 and 0 to 30% synthetic resin fibers by weight of the liquid retaining layer 7.

Where the synthetic resin fibers of the first and the second liquid permeable layers 5, 6 and the liquid retaining layer 7 contain a low-melting point resin, such as PE and low-melting PP, and are heat-fusible per se, on the other hand, fibers may be fusion-bonded together by a bonding force caused by fusion of the synthetic resin fibers, in place of, or in addition to, fiber-to-fiber adhesion through the emulsion binder or the fibrous binder.

At the boundary 8 of the liquid absorbent sheet 1, constituent fibers of the first liquid permeable layer 5 and constituent fibers of the liquid retaining layer 7 are bonded together in a slightly intermingled state. This is also true at the boundary 9.

The liquid absorbent sheet 1 preferably has a water absorption capacity equal to or greater than 300 g/m² and a water retention capacity equal to or greater than 200 g/m². The water absorption capacity and water retention capacity may be determined as follows. A sample of 140×60 mm is cut out of the liquid absorbent sheet 1, immersed in a saline (NaCl content of 0.9 wt. %) for three minutes, and after removal from the saline, allowed to stand still for five minutes on a 10 mesh wire net. The water absorption capacity may be calculated from a saline content of the sample at this time. Also after immersion in the saline for three minutes, the sample is placed on a plane and subjected to a pressure of 490 Pa for two minutes. The water retention capacity may be calculated from a saline content of the sample at this time.

Next, a method for manufacturing the liquid absorbent sheet 1 will be described with reference to FIG. 4.

In a manufacturing equipment shown in FIG. 4, a net-like endless conveyor belt 33 turns on rolls 31, 32. Above the endless conveyor belt 33, first, second and third supply ducts 34, 35, 36 are arranged in downstream order so as to form a fibrous web on the endless conveyor belt 33 by an air-laid process. In the air-laid process, opened staple fibers are let fall in the air from the supply ducts 34, 35, 36 and deposited on the endless conveyor belt 33.

Firstly from the first supply duct 34, a mixture of fibers (70-100 wt. % synthetic resin fibers and 0-30 wt. % cellulosic fibers) is supplied and deposited on the endless conveyor belt 33 to form a second liquid permeable fibrous web 41 (which becomes the second liquid permeable layer 6 in the finished product). Secondly from the second supply duct 35, a mixture of fibers (70-100 wt. % cellulosic fibers and 0-30 wt. % synthetic resin fibers) is supplied and deposited on the second liquid permeable fibrous web 41 to form a liquid retaining fibrous web 42 (which becomes the liquid retaining layer 7 in the finished product). Thirdly from the third supply duct 36, a mixture of fibers (70-100 wt. % synthetic resin fibers and 0-30 wt. % cellulosic fibers) is supplied and deposited on the liquid retaining fibrous web 42 to form a first liquid permeable fibrous web 43 (which becomes the first liquid permeable layer 5 in the finished product).

Thus, there is obtained a multi-ply fibrous web 40 in which the three fibrous webs 41, 42, 43 are deposited in order of mention. The multi-ply fibrous web 40 is then pressed between press rolls 45, 46. After pressing, a binder, such as an acrylic binder, is supplied from a binder nozzle 49 while the multi-ply fibrous web 40 is conveyed on a conveyor belt 48 turning on a roll 47. Then, the multi-ply fibrous web 40 is dried in a drying chamber 50, wherein the fibers are bonded together due to evaporation of a solvent of the binder. It should be noted that the multi-ply fibrous web 40 may be pressed after application of the binder with the binder nozzle 49 located upstream of the press rolls 45, 46.

When fusion-bonding the synthetic resin fibers (with or without the use of the binder), the press rolls 45, 46 may be replaced by heating rolls, wherein the synthetic resin fibers can be fusion-bonded to each other or to the cellulosic fibers by melting their surfaces by heat of the heating rolls. When adding a fibrous binder to each layer, such a fibrous binder may be contained in each of the fibrous webs 41, 42, 43 deposited on the endless conveyor belt 33 and melted in the heating step with the use of the heating rolls.

The liquid absorbent sheet 1 thus manufactured has a sufficient sheet strength. At the boundaries 8, 9, moreover, constituent fibers of the upper and lower layers are bonded together in a slightly intermingled state, which prevents the conventional interface that would be formed by bonding preformed sheets. Therefore, the boundaries 8, 9 do not resist transfer of liquid from the upper layer to the lower layer.

Here, since the second liquid permeable fibrous web 41, the liquid retaining fibrous web 42 and the first liquid permeable fibrous web 43, which are dry laid by an air-laid process, are immediately pressed between the press rolls 45, 46, as shown in FIG. 4, constituent fibers of the upper and lower layers are not excessively intermingled at the boundaries 8, 9. In the finished liquid absorbent sheet 1, therefore, the first and the second liquid permeable layers 5, 6 can be distinguished to some extent from the liquid retaining layer 7. As a result, the functions of the individual layers can be clearly distinguished from each other even in such a thin sheet.

The liquid absorbent sheet 1, whose fibers are bonded together after pressed between the press rolls 45, 46, has a relatively high bending stiffness. In addition, the first and the second surfaces 3, 4 are smoothed by the press rolls 45, 46.

As shown in FIG. 1, the liquid absorbent sheet 1 may be cut into a size corresponding to the food tray 2 and a food to be housed in the food tray 2 and laid on the bottom panel of the food tray 2.

A drip exuding from a fresh food (not shown) on the liquid absorbent sheet 1 is supposed to be first applied to the first surface 3. This drip passes through the first liquid permeable layer 5 via voids between fibers under force of gravity or due to capillary action and is then absorbed and retained by the liquid retaining layer 7 due to its hydrophilicity. Since the liquid retaining layer 7 is mainly comprised of cellulosic fibers having a strong hydrophilicity and has a high density, the drip applied to the first liquid permeable layer 5 can be rapidly absorbed and retained by the liquid retaining layer 7.

The drip retained in the liquid retaining layer 7 is effectively prevented from returning and adhering to the food because the first liquid permeable layer 5 mainly of synthetic resin fibers is disposed between the food and the liquid retaining layer 7. The first liquid permeable layer 5 with a great number of voids between fibers is also effective in preventing the drip from remaining on the first surface 3. Therefore, the fresh food such as meat or fresh fish on the liquid absorbent sheet 1 can be kept from discoloring or deteriorating due to contact with the drip. In addition, bacteria, which tends to grow in the drip, can be prevented from adhering to the food, which also aids in maintaining freshness of the fresh food for a long period of time.

Although the second liquid permeable layer 6 is in contact with the bottom panel of the food tray 2, since the cellulosic fibers of the liquid retaining layer 7 have a stronger hydrophilicity than the synthetic resin fibers of the second liquid permeable layer 6 and the liquid retaining layer 7 has a higher fiber density than the second liquid permeable layer 6, the drip retained in the liquid retaining layer 7 does not easily transfer to the second liquid permeable layer 6 and adhere to the bottom panel of the food tray 2. Therefore, the tray surface can be kept clean and the product can be prevented from being disfigured by meat blood or the like flowing out onto the bottom panel of the tray.

Because the liquid permeable layers 5, 6 are provided on opposite sides of the liquid absorbent sheet 1, the liquid absorbent sheet 1 may be used regardless of which one of the first and the second surfaces 3, 4 is directed downward, which makes it unnecessary to observe the correct side in handling.

FIG. 3 shows a liquid absorbent sheet 101 according to a second embodiment of the present invention.

The liquid absorbent sheet 101 is also a single nonwoven fabric, but is constructed of only two layers: liquid permeable layer 105 and liquid retaining layer 107. The liquid permeable layer 105 has the same fiber makeup as the first and the second liquid permeable layers 5, 6, while the liquid retaining layer 107 has the same fiber makeup as the liquid retaining layer 7. The liquid absorbent sheet 101 may be manufactured by the manufacturing equipment of FIG. 4 without using the first supply duct 34 or the third supply duct 36.

Preferred basis weights of the liquid permeable layer 105 and the liquid retaining layer 107, preferred water absorption capacity and water retention capacity of the liquid absorbent sheet 101, and so on are equal to those of the first embodiment.

The liquid absorbent sheet 101 may be used with a second surface 104 (the liquid retaining layer 107) directed toward the bottom panel of the food tray 2 so that a first surface 103 (the liquid permeable layer 105) can face a food. This liquid absorbent sheet 101 also prevents a drip from returning and adhering to the food.

It should be noted that the liquid absorbent sheet according to the present invention is suitable not only for use in absorbing a food drip; as has been described hereinabove, but also for use in a pet toilet sheet, a panty liner, a thin sanitary napkin and the like. In this case, urine, blood or vaginal discharge absorbed in the liquid retaining layer 7, 107 can be prevented from returning to a surface directed to the wearer's body.

Although the present invention has been illustrated and described with respect to exemplary embodiments thereof, it should be understood by those skilled in the art that the foregoing and various other changes, omission and additions may be made therein and thereto, without departing from the spirit and scope of the present invention. Therefore, the present invention should not be understood as limited to the specific embodiments set out above but should be understood to include all possible embodiments which can be embodied within a scope encompassed and equivalent thereof with respect to the feature set out in the appended claims.

1. A liquid absorbent sheet for absorbing and retaining liquid, the liquid absorbent sheet being a single nonwoven fabric, comprising:
a liquid permeable layer; and
a liquid retaining layer,
wherein the liquid permeable layer and the liquid retaining layer are inseparably but distinguishably formed, the liquid permeable layer being exposed externally on one side of the nonwoven fabric, the liquid retaining layer lying beneath the liquid permeable layer for absorbing and retaining liquid,
the liquid permeable layer contains at least 70% synthetic resin fibers by weight, and the liquid retaining layer contains at least 70% cellulosic fibers by weight,
the fibers of the nonwoven fabric are bonded together by an adhesive force of a binder and/or a bonding force caused by fusion of the synthetic resin fibers.

2. The liquid absorbent sheet of claim 1, wherein the liquid retaining layer lies within the nonwoven fabric with two liquid permeable layers exposed externally on opposite sides of the nonwoven fabric.

3. The liquid absorbent sheet of claim 1, wherein the liquid retaining layer is exposed externally on the other side of the nonwoven fabric.

4. The liquid absorbent sheet of claim 1, wherein the liquid retaining layer has a higher fiber density than the liquid permeable layer.

5. A method for manufacturing a liquid absorbent sheet comprising the steps of:
(a) depositing a multi-ply fibrous web with a liquid permeable fibrous web containing at least 70% synthetic resin fibers by weight and a liquid retaining fibrous web containing at least 70% cellulosic fibers by weight;
(b) pressing the multi-ply fibrous web; and
(c) before or after the step (b), bonding constituent fibers of the multi-ply fibrous web by an adhesive force of a binder and/or a bonding force caused by fusion of the synthetic resin fibers.

6. The method of claim 5, further comprising a step (d) of drying the multi-ply fibrous web after the steps (a), (b) and (c).

7. The method of claim 5, wherein in the step (a), the fibers of the liquid permeable fibrous web and the fibers of the liquid retaining fibrous web are deposited by an air-laid process.

8. The method of claim 5, wherein in the step (a), the multi-ply fibrous web is formed by depositing the liquid retaining fibrous web on the liquid permeable fibrous web and further depositing another liquid permeable fibrous web on the liquid retaining fibrous web.
